(19) United States
(12) Patent Application Publication
(10) Pub. No.: US 2006/0134004 A1
(43) Pub. Date: Jun. 22, 2006

(54) Methods and apparatus for detection of carotenoids in macular tissue

(75) Inventors: Werner Gellermann, Salt Lake City, UT (US); Mohsen Sharifzadeh, Salt Lake City, UT (US); Igor V. Ermakov, Salt Lake City, UT (US); Paul S. Bernstein, Salt Lake City, UT (US)

Correspondence Address: HOLME ROBERTS & OWEN, LLP, 299 SOUTH MAIN, SUITE 1800, SALT LAKE CITY, UT, 84111, US

(73) Assignee: The University of Utah

(21) Appl. No.: 11/018,403

(22) Filed: Dec. 21, 2004

(51) Int. Cl. A61K 49/00; A61B 10/00

(52) U.S. Cl. 424/9.6; 600/315

(57) ABSTRACT

Methods and apparatus are provided for the noninvasive detection and measurement of macular pigments such as carotenoids in macular tissue. In one technique, lipoftiscin autofluorescence spectroscopy is utilized for macular pigment measurements. In autofluorescence spectroscopy, the emission of lipoftiscin is excited at two wavelengths: one wavelength that overlaps both the macular pigment and lipofuscin absorption and another wavelength that lies outside the macular pigment absorption range but that still excites the lipofuscin emission. The macular pigment absorption is then derived from the different lipoftiscin emission intensities in the macula and peripheral retina. In another technique, both autofluorescence spectroscopy, as described above, and resonance Raman spectroscopy are used to identify and quantify the presence of carotenoids in macular tissue. In using resonance Raman spectroscopy, laser light is directed onto the eye tissue and the scattered light is then spectrally filtered and detected. The frequency difference between the laser light and the Raman scattered light is known as the Raman shift. The magnitude of the Raman shift is an indication of the type of chemical present, and the intensities of the Raman signal peaks correspond directly to the chemical concentration.

### BACKGROUND OF THE INVENTION

1. Field of the Invention

The present invention relates generally to techniques for measuring levels of chemical compounds found in biological tissue. More specifically, the invention relates to methods and apparatus for the noninvasive detection and measurement of levels of carotenoids and related chemical substances in macular tissue.

2. Relevant Technology

Carotenoids are important ingredients for the anti-oxidant defense system of the human body. Numerous epidemiological and experimental studies have shown that a higher dietary intake of carotenoids may protect against cancer, age-related macular degeneration, pre-mature skin aging, and other pathologies associated with oxidative cell damage.

The standard methods that have been used for measuring carotenoids are through high-performance liquid chromatography (HPLC) techniques. Such techniques require that large amounts of tissue sample be removed from the patient for subsequent analysis and processing, which typically takes at least 24 hours to complete. In the course of these types of analyses, the tissue is damaged, if not completely destroyed. Therefore, a noninvasive and more rapid technique for measurement is preferred.

There is considerable interest to measure macular carotenoid levels noninvasively in the population to determine whether or not low levels of macular pigments are associated with increased risk of age-related macular degeneration (AMD). Currently, the most commonly used noninvasive method for measuring human macular pigment (MP) levels is a subjective psychophysical heterochromatic flicker photometry test involving color intensity matching of a light beam aimed at the fovea and another aimed at the perifoveal area. However, this method is rather time consuming and requires an alert, cooperative subject with good visual acuity. This method can also exhibit a high intrasubject variability when macular pigment densities are low or if significant macular pathology is present. Thus, the usefulness of this method for assessing macular pigment levels in the elderly population most at risk for AMD is severely limited. Nevertheless, researchers have used flicker photometry to investigate important questions such as variation of macular pigment density with age and diet.

A number of objective techniques for the measurement of MP in the human retina have been explored recently as alternatives to the subjective psychophysical tests. The underlying optics principles of these techniques are either based on fundus reflection or fundus fluorescence (autofluorescence) spectroscopy. In traditional fundus reflectometry, which uses a light source with a broad spectral continuum, reflectance spectra of the bleached retina are separately measured for fovea and perifovea. The double-path absorption of MP is extracted from the ratio of the two spectra by reproducing its spectral shape in a multi-parameter fitting procedure using appropriate absorption and scattering profiles of the various fundus tissue layers traversed by the source light. One of the imaging variants of fundus reflectometry uses a TV-based imaging fundus reflectometer with sequential, narrow bandwidth light excitation over the visible wavelength range and digitized fundus images. Another powerful variant uses a scanning laser ophthalmoscope, employing raster-scanning of the fundus with discrete laser excitation wavelengths to produce highly detailed information about the spatial distribution of MP (and photopigments).

In autofluorescence spectroscopy, lipofuscin in the retinal pigment epithelium is excited with light within and outside the wavelength range of macular pigment absorption, but within the absorption range of lipofuscin. This can be realized, for example, with 488 nm and 532 nm light sources, respectively. The blue (488 nm) wavelength is absorbed both by macular pigment and lipofuscin; the green (532 nm) wavelength is absorbed only by lipofascin. By measuring the lipofliscin fluorescence intensity levels for the foveal and peripheral retina regions, I (fovea) and I (peri), respectively, for both excitation wavelengths, an estimate of the single-pass absorption of MP can be obtained. Specifically, the optical density (O.D.) of the macular pigment is given by the expression
O.D.=c{log [I(fovea, 532 nm)/I(fovea, 488 nm)]−log [I(peri,532 nm)/I(peri, 488 nm)]} (1),
where c is a constant factor that compensates for the different magnitudes of the extinction coefficients for the two different wavelengths (the factor is ˜1.2 for the set of wavelengths 488 nm and 532 nm). A disadvantage of the autofluoresecence technique is its low specificity. In principle, any absorber absorbing in the same wavelength range as the MP can artifactually attenuate the lipofuscin excitation, and thus lead to an erroneous mapping of the MP distribution and its concentration levels. This could be a serious drawback, particularly in the presence of retinal pathology (e.g. drusen, bleeding vessels, etc).

Raman spectroscopy is a highly specific form of vibrational spectroscopy that can be used to noninvasively identify and quantify chemical compounds. Carotenoid molecules are especially suitable for Raman measurements because they can be excited with light overlapping their visible absorption bands, and under these conditions, they exhibit a very strong Resonance Raman scattering (RRS) response, with an enhancement factor of about five orders of magnitude relative to non-resonant Raman spectroscopy. This allows one to non-invasively detect the characteristic vibrational energy levels of the carotenoids through their corresponding spectral “fingerprint” signature, even in complex biological systems.

A disadvantage of Raman spectroscopy is the inability to easily compensate for the absorption effect of ocular media. Strong Raman signals can only be obtained from the central macular area, but not from peripheral areas, due to the rapid drop of MP levels towards the periphery. Therefore, the optical density of the MP in the central area cannot simply be calculated by comparing the intensities of peripheral and macular areas. However, it is possible to remedy this drawback by using correction factors derived from other measurements. For example, it is possible to determine the attenuation effect of the major attenuating ocular component, the eye lens, by measuring the reflection of blue/green light from the anterior and posterior surfaces of the lens (Purkinje images).

A noninvasive method for the measurement of carotenoid levels in the macular tissue of the eye is described in U.S. Pat. No. 5,873,831, the disclosure of which is incorporated by reference herein, in which levels of carotenoids and related substances are measured by resonance Raman spectroscopy. In this technique, nearly monochromatic light is incident upon the sample to be measured, and inelastically scattered light which is of a different frequency than the incident light is detected and measured. The frequency shift between the incident and scattered light is known as the Raman shift, and this shift corresponds to an energy which is the fingerprint of the vibrational or rotational energy state of certain molecules. Typically, a molecule exhibits several characteristic Raman active vibrational or rotational energy states, and the measurement of the molecule's Raman spectrum thus provides a fingerprint of the molecule, i.e., it provides a molecule-specific series of spectrally sharp vibration or rotation peaks. The intensity of the Raman scattered light corresponds directly to the concentration of the molecule(s) of interest.

Another noninvasive method for the measurement of carotenoids and related chemical substances in biological tissue by resonance Raman spectroscopy is disclosed in U.S. Pat. No. 6,205,354 B1, the disclosure of which is incorporated by reference herein. This technique provides for a rapid, accurate, and safe determination of carotenoid levels which in turn can provide diagnostic information regarding cancer risk, or can be a marker for conditions where carotenoids or other antioxidant compounds may provide diagnostic information. In this technique, laser light is directed upon the area of tissue which is of interest such as the skin. A small fraction of the scattered light is scattered inelastically, producing the carotenoid Raman signal which is at a different frequency than the incident laser light, and the Raman signal is collected, filtered, and measured. The resulting Raman signal can be analyzed such that the background fluorescence signal is subtracted and the results displayed and compared with known calibration standards.

### SUMMARY OF THE INVENTION

The present invention is directed to methods and apparatus for the noninvasive detection and measurement of macular pigments such as carotenoids and related chemical substances in macular tissue. In one aspect of the invention, lipofuscin autofluorescence spectroscopy is utilized for macular pigment measurements. In autofluorescence spectroscopy, the emission of lipofuscin is excited at two wavelengths: one wavelength that overlaps both the macular pigment and lipofuscin absorption and another wavelength that lies outside the macular pigment absorption range but that still excites the lipofuscin emission. The macular pigment absorption is then derived from the logarithms of the lipofuscin emission intensities in the macular region and peripheral retina obtained for both wavelengths, according to equation (1).

In another aspect of the invention, both autofluorescence spectroscopy and resonance Raman spectroscopy are used to identify and quantify the presence of carotenoids and similar substances in macular tissue. In this combined technique, the autofluorescence spectroscopy is used in a similar manner as described above. In using resonance Raman spectroscopy, laser light is directed onto the eye tissue and the scattered light is then spectrally filtered and detected. Most of the scattered light is scattered elastically. A small remainder of the light is scattered inelastically, and is therefore of different frequencies than the incident laser light. This inelastically scattered light forms the Raman signal. The frequency difference between the laser light and the Raman scattered light is known as the Raman shift and is typically measured as a difference in wave numbers. The magnitude of the Raman shifts is an indication of the type of chemical present, and the intensities of the Raman signal peaks correspond directly to the chemical concentration.

In a method of the invention that uses autofluorescence spectroscopy, a first light source and a second light are provided that emit different wavelengths of light. Light from the first light source overlaps in wavelength the absorption spectrum of macular pigment and the absorption of lipofuscin. The second light source has a longer wavelength compared to the first light source, such that its wavelength is outside the absorption range of macular pigment but still within the long-wavelength shoulder of the lipofuscin absorption. Both light sources have the same illumination spot size and are sequentially directed onto the retina of the eye such that the macula of the subject is centered in the illuminated spots. The light emitted from the retinal tissue is collected for both excitation light sources, with the collected light comprising lipofuscin emissions from the macular and peripheral retinal areas. The lipofliscin emission intensities will be attenuated in the macular region of the retina with respect to the peripheral retina when using the first light source, since the excitation light is absorbed by macular pigment and the lipofuscin emission is therefore weaker in the macular area as compared to the periphery. The lipofuscin intensities will be similar in the macula and peripheral areas when using the second light source since there is no absorption of the excitation light by macular pigment in this case. Any difference in intensities can only stem from an uneven distribution of lipofuscin throughout the retina, or from spatially differing absorber distributions of other compounds such as melanin. For example, there could be less lipofuscin in the macular region and more in peripheral areas, or there could be more melanin in some areas than others. The lipofuscin fluorescence intensity distributions obtained with the second light source therefore are useful to correct the intensity distributions in the macular and peripheral areas obtained with the first light source. The lipofuscin emission intensity distributions obtained for the two excitation wavelengths are quantified, and the macular pigment levels in the macular tissue are determined according to equation (1) from the logarithms of the lipofuscin emission intensities for the two excitation wavelengths measured at central and peripheral retinal locations.

In a method of the invention that uses autofluorescence spectroscopy and resonance Raman spectroscopy, two light sources are preferably provided that generate light at two wavelengths that each produce autofluorescence lipofuscin emission but that are chosen such that only one of the light sources is attenuated by macular pigment. Light from these light sources is directed onto macular tissue of an eye for which macular pigment levels are to be measured, and the lipofuscin emission intensities are collected in a first optical channel, the collected light comprising lipoftiscin emissions from macular and peripheral retinal areas for the two excitation wavelengths. The lipofuscin emission intensities are then detected and quantified at the first and second wavelengths. The macular pigment levels in the macular tissue are then determined again according to (equation 1). For the excitation light source which overlaps the absorption of macular pigment, the light scattered from the macular tissue area is collected in a second optical channel, the scattered light including elastically and inelastically scattered light, with the inelastically scattered light producing a Raman signal corresponding to carotenoids in the tissue. The elastically scattered light is filtered out, and the intensity of the Raman signal is quantified.

These and other features of the present invention will become more fully apparent from the following description and appended claims, or may be learned by the practice of the invention as set forth hereinafter.

### BRIEF DESCRIPTION OF THE DRAWINGS

In order to illustrate the above and other features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a graphical diagram of the absorption spectra, molecular structure, and energy level scheme of major carotenoid species found in human tissue, including β-carotene, zeaxanthin, lycopene, lutein and phytofluene.

FIG. 2 is a graphical diagram of the resonance Raman spectra of β-carotene, zeaxanthin, lycopene, lutein, and phytofluene solutions, showing the three major “spectral fingerprint” Raman peaks of carotenoids originating from rocking motions of the methyl components (C—CH₃) and stretch vibrations of the carbon-carbon single bonds (C—C) and double bonds (C═C).

FIGS. 3A-3F are graphs of the absorption spectra and resonance Raman responses for solutions of β-carotenes, lycopenes, and a mixture of both.

FIG. 4 is a schematic representation of the retinal layers that participate in light absorption, transmission, and scattering of excitation and emission light, including the ILM (inner limiting membrane), NFL (nerve fiber layer), HPN (henle fiber, plexiform, and nuclear layers), PhR (photoreceptor layer), and RPE (retinal pigment epithelium).

FIG. 5 is a schematic depiction of an apparatus according to the invention that can be employed for measuring macular pigments using autofluorescence spectroscopy.

FIG. 6 is a schematic depiction of one embodiment of an apparatus according to the invention that can be employed for simultaneous Raman and autofluorescence-based detection of macular pigments.

FIG. 7 is a schematic depiction of another embodiment of an apparatus according to the invention that can be employed for simultaneous Raman and autofluorescence-based detection of macular pigments.

FIG. 8 is a graph of the absorption and emission spectra of A2E, the main fluorophore of lipofuscin, dissolved in methanol and shown as a solid curve. The dashed curve represents the absorption of the macular pigments, showing that there is strong spectral overlap between the MP absorption and the A2E absorption.

FIG. 9 includes photomicrographs of the retina of a human volunteer subject, showing image a obtained by measuring the reflection of white light, image b which is a lipofuscin fluorescence digital fundus image obtained under 488 nm illumination, and image c which is the lipofuscin fluorescence digital fundus image obtained under 532 nm illumination.

FIGS. 10A-10D includes photomicrographs of the retina of four human volunteer subjects (A-D) showing digital subtraction images of spatial MP distributions, line plots of transmissions, and line plots of absorptions for the subjects A-D.

FIGS. 11A-11D display pseudocolor topographical maps showing MP distributions in four volunteer subjects A-D.

FIG. 12 is a bar graph of MP concentrations for six volunteer subjects A-F, showing the total pigment concentration of each individual, obtained by integrating each individual's distribution over its area.

FIG. 13 is a bar graph showing a comparison of MP intensities, measured for four subjects A-D by autofluorescence and resonance Raman detection techniques.

FIG. 14 shows schematically the lipofuscin emission intensity maps (autofluorescence images) obtained in a retinal region centered around the macula, obtained with the autofluorescence technique of the invention.

FIG. 15 is a graph of MP optical densities obtained from autofluorescence images (pixel intensity maps) for a series of long-wavelength pass filters (cut-on wavelength λ_{c}) used to block off part of the lipofuscin emission range.

### DETAILED DESCRIPTION OF THE INVENTION

The present invention is directed to methods and apparatus for the noninvasive detection and measurement of macular pigments such as carotenoids and related chemical substances in macular tissue. In particular, the present method and apparatus make possible the rapid, noninvasive, and quantitative measurement of the concentration of carotenoids, as well as their isomers and metabolites, in macular tissue. The invention can be used in a direct and quantitative optical diagnostic technique, which uses low intensity illumination of intact tissue and provides high spatial resolution, allowing for precise quantification of the carotenoid levels in the tissue.

In one aspect of the invention, lipofliscin fluorescence excitation spectroscopy (“autofluorescence or AF spectroscopy”) is utilized for MP measurements. In AF spectroscopy, the emission of lipofuscin, located in the retinal pigment epithelial layer, is excited at two wavelengths: one wavelength that overlaps both the MP and lipofuscin absorption and another, longer wavelength, that lies outside the MP absorption range but that still excites the lipofuscin emission. The MP absorption is then derived from the logarithms of lipofuscin emission intensities obtained for macular and peripheral retinal areas for both excitations (according to equation 1).

In the present technique for AF-based MP measurements, a simple imaging approach is used based on an imaging CCD camera, two laser light sources, and a light delivery and collection module. Digital MP images of a subject are indirectly recorded by detecting the lipofuscin fluorescence of the retinal pigment epithelium over a retinal area that includes the macular region upon sequential excitation with 488 nm and 532 nm light, and the spatial extent of MP and its topographic concentration distribution is obtained by digital image processing (taking into account the differing pixel intensity maps; see equation (1)).

In another aspect of the invention, AF spectroscopy and resonance Raman spectroscopy are combined in order to identify and quantify the presence of carotenoids and similar substances in MP. This technique allows one to measure as accurately as possible the macular pigment existing in the retina of a subject's eye. In particular, this technique of the invention provides a simultaneous image of the spatial distribution details (i.e., extent, symmetries, discontinuities, topology in general) and the integrated concentration of the pigments (“quantitative imaging”).

Previous results on MP distributions in excised retinas point to the fact that different individuals have different MP distributions as well as absolute levels. For example, one person could have a narrow MP distribution with a very high or low central concentration, while another one could have a much wider concentration and a relatively low/high central pigment concentration. The integrated concentrations in these individuals could be very similar in some cases, and an integral measurement alone would not be able to reveal any difference. Knowledge of the spatial differences, however, as well as the absolute MP level concentrations is important to help understand the development and progression of age-related macular degeneration, the leading cause of irreversible blindness in the elderly. The combined autofluorescence and Raman based technique of the present invention provides a unique way to measure both aspects of MPs simultaneously. This technique combines the imaging capability of autofluorescence with the high molecular specificity of Raman spectroscopy.

In autofluorescence based spectroscopy, the MP levels and their spatial distribution are determined indirectly by comparing the lipofuscin emission originating in the retinal pigment epithelium under blue and green light excitation. In both cases, a large area of the retina is illuminated that contains the MP-rich macular region and an MP-poor peripheral region. The optical density of the MP is determined from the ratio of the lipofuscin emission intensities measured in the macular and peripheral regions, respectively, under both excitations, according to equation (1). An advantage of autofluorescence based MP measurements is the relatively high light level of the fluorescence signal, which allows one to work with relatively short exposure times and to record the emission over a large retinal area. Also, it is possible with this technique to eliminate the influence of ocular media (e.g., lens opacities, etc.) on the MP levels, since their absorption/scattering contributions cancel out when comparing macular and peripheral light levels.

An advantage of Raman spectroscopy is its extremely high specificity, since it is capable of distinguishing between molecules by measuring their sharp vibrational levels. In general, different molecules have different vibrational levels. By using Raman spectroscopy it is easily possible to filter out unwanted responses and to only record the vibrational response of the molecules of interest. Since the Raman response signal of the molecules of interest is generally proportional to their concentration, at least for physiological concentration levels, it is possible to directly measure the concentration of the molecules of interest.

Thus, the autofluorescence/Raman based technique of the present invention combines the strength of autofluorescence spectroscopy with the strength of Raman spectroscopy. By using two detection channels, it is possible to record simultaneously an integral concentration score of the MP concentration existing in the macular region determined by high-specificity Raman spectroscopy, and a spatial map of MP determined via lipofuscin excitation spectroscopy. The Raman-based MP concentration is used to calibrate the concentration of the autofluorescence-based MP image recorded with the other detection channel, or vice versa, making sure that both measurements agree.

Further details of the MP measurement techniques of the present invention are discussed hereafter.

Optical Properties and Resonance Raman Scattering of Carotenoids

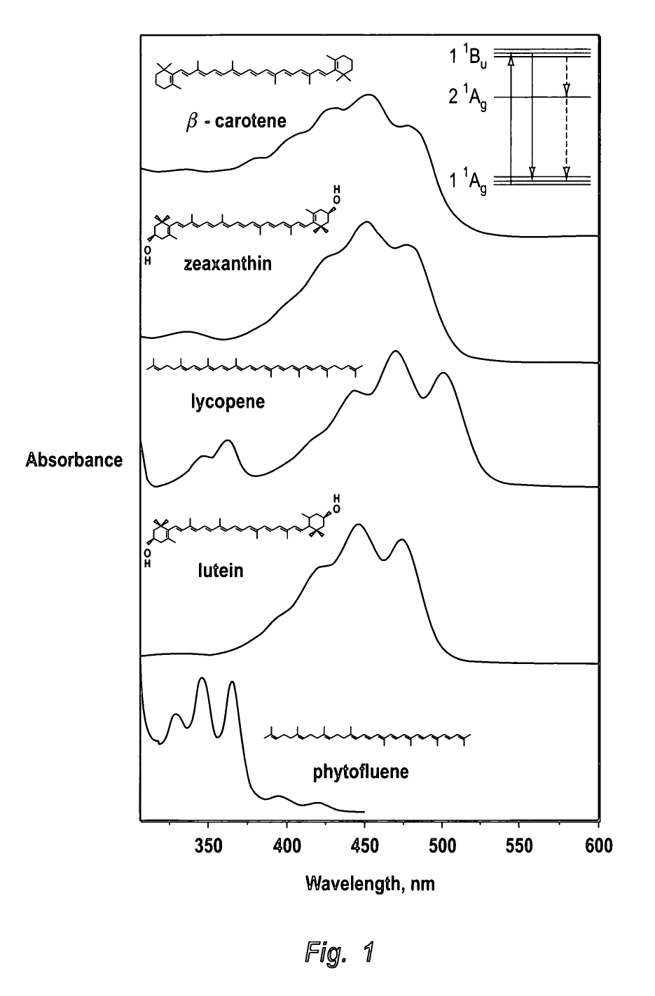

Carotenoids are n-electron conjugated carbon-chain molecules and are similar to polyenes with regard to their structure and optical properties. Distinguishing features are the number of conjugated carbon double bonds (C═C bonds), the number of attached methyl side groups, and the presence and structure of attached end groups. The molecular structures of some of the most important carotenoid species found in human tissue, along with their absorption spectra and energy level scheme, are shown in the diagram of FIG. 1. They include β-carotene, zeaxanthin, lycopene, lutein and phytofluene, which feature an unusual even parity excited state. As a consequence, absorption transitions are electric-dipole allowed in these molecules but spontaneous emission is forbidden. The electronic absorptions are strong in each case, occur in broad bands (˜100 nm width), and shift to longer wavelength with increasing number of effective conjugation length of the corresponding molecule. The absorption of phytofluene (five conjugated C═C bonds, respectively) is centered at ˜340 nm, and lycopene (11 bonds) peaks at ˜450 nm. All show a clearly resolved vibronic substructure due to weak electron-phonon coupling, with spacing of ˜1400 cm⁻¹. Strong electric-dipole allowed absorption transitions occur between the molecules' delocalized π-orbitals from the 1 ¹A_{g} singlet ground state to the 1 ¹Bᵤ singlet excited state (see inset of FIG. 1).

All carotenoid molecules feature a linear, chain-like conjugated carbon backbone including alternating carbon single (C—C) and double bonds (C═C) with varying numbers of conjugated C═C double bonds, and a varying number of attached methyl side groups. Beta-carotene, lutein, and zeaxanthin feature additional ionone rings as end groups. In β-carotene and zeaxanthin, the double bonds of these ionone rings add to the effective C═C double bond length of the molecules. Lutein and zeaxanthin have an OH group attached to the ring. Lycopene has 11 conjugated C═C bonds, β-carotene has 11, zeaxanthin has 11, lutein has 10, and phytofluene has 5. The absorptions of all species occur in broad bands in the blue/green spectral range, with the exception of phytofluene, which as a consequence of the shorter C═C conjugation length absorbs in the near UV. Also, a small (˜10 nm) spectral shift exists between the lycopene and lutein absorptions.

In all carotenoids, optical excitation within the absorption band leads to only very weak luminescence bands. The extremely low quantum efficiency of the luminescence is caused by the existence of a second excited singlet state, a 2 ¹A_{g} state, which lies below the 1 ¹Bᵤ state (see FIG. 1 inset). Following excitation of the 1 ¹Bᵤ state, the carotenoid molecule relaxes very rapidly, within ˜200-250 fs, via nonradiative transitions, to this lower 2 ¹A_{g} state from which electronic emission to the ground state is parity-forbidden (dashed, downward pointing arrows in inset of FIG. 1). The low 1 ¹Bᵤ→1 ¹A_{g} luminescence efficiency (10⁻⁵-10⁻⁴) and the absence of 2 ¹A_{g}→1 ¹A_{g} fluorescence of the molecules allows one to detect the RRS response of the molecular vibrations (shown as solid, downward pointing arrow in inset of FIG. 1) without potentially masking fluorescence signals. Specifically, resonance Raman spectroscopy detects the stretching vibrations of the polyene backbone as well as the methyl side groups.

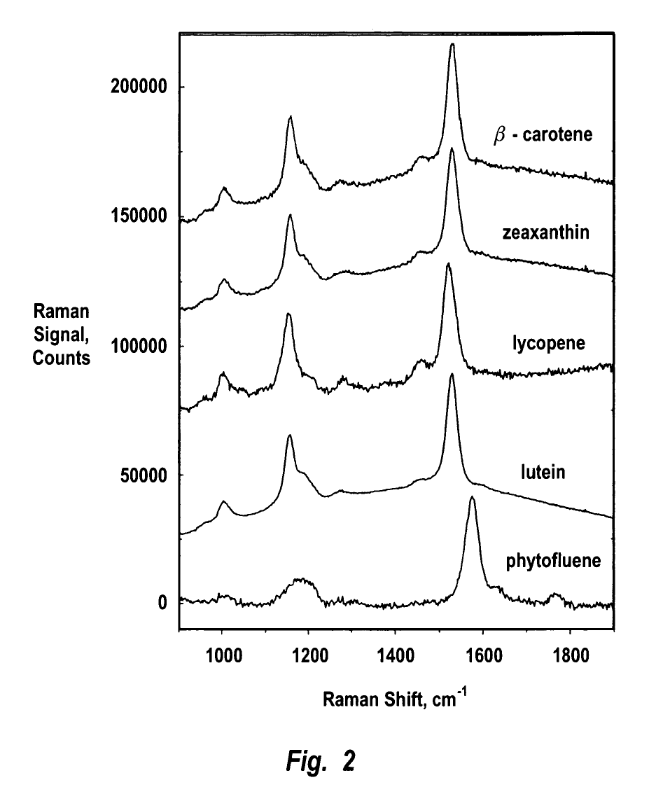

Tetrahydrofuran solutions of the carotenoids depicted in FIG. I were used to obtain the RRS spectra shown in FIG. 2. Beta-carotene, zeaxanthin, lycopene, and lutein all have strong and clearly resolved Raman signals superimposed on a weak fluorescence background, with three prominent Raman Stokes lines appearing at ˜1525 cm⁻¹ (C═C stretch vibration), 1159 cm⁻¹ (C—C stretch vibration), and 1008 cm⁻¹ (C—CH₃ rocking motions). In the shorter-chain phytofluene molecule, only the C═C stretch appears, and it is shifted significantly to higher frequencies (by ˜40 cm⁻¹). The large contrast between Raman response and broad background signal is due to the inherently weak fluorescence of carotenoids.

Raman scattering does not require resonant excitation, in principle, and is therefore useful to simultaneously detect the vibrational transitions of all Raman active compounds in a given sample. However, off-resonant Raman scattering is a very weak optical effect, requiring intense laser excitation, long signal acquisition times, and highly sensitive, cryogenically cooled detectors. Also in biological systems the spectra tend to be very complex due to the diversity of compounds present. The scenario changes drastically if the compounds exhibit absorption bands due to electronic dipole transitions of the molecules, particularly if these are located in the visible wavelength range. When illuminated with monochromatic light overlapping one of these absorption bands, the Raman scattered light will exhibit a substantial resonance enhancement. In the case of carotenoids, 488 nm argon laser light provides an extraordinarily high resonant enhancement of the Raman signals on the order of 10⁵. No other biological molecules found in significant concentrations in human tissues exhibit similar resonant enhancement at this excitation wavelength, so in vivo carotenoid RRS spectra are remarkably free of confounding Raman responses.

Raman scattering is a linear spectroscopy, meaning that the Raman scattering intensity (I_{S}) scales linearly with the intensity of the incident light (I_{L}), as long as the scattering compound can be considered as optically thin. Furthermore, at fixed incident light intensity, the Raman response scales with the population density of the scatters N(Eᵢ) in a linear fashion with the Raman scattering cross section σ_{R}(i→f) (a fixed constant whose magnitude depends on the excitation and collection geometries) as long as the scatterers can be considered as optically thin. Here, (i) designates the initial energy state, and (f) the final energy state. This phenomenon is described by equation 2.
Iₛ=N(Eᵢ)×σ_{R}×I_{L} (2)
In optically thick media, as in geometrically thin but optically dense tissue, a deviation from the linear Raman response of Iₛ versus concentration N can occur, of course—for example due to self absorption of the Stokes Raman line by the strong electronic absorption. In general, this can be taken into account, at least over a limited concentration range, by calibrating the Raman response with suitable tissue phantoms.

RRS spectroscopy has an additional advantage over ordinary Raman spectroscopy in the possibility to influence the Raman response by judicious choice of the excitation wavelength. This allows one to selectively enhance the Raman response of one carotenoid species over another one in a mixture of compounds. For example, exciting a mixture of phytofluene and lutein at 450 nm would only result in a RRS response from lutein, thus allowing to selectively quantify lutein in this mixture.

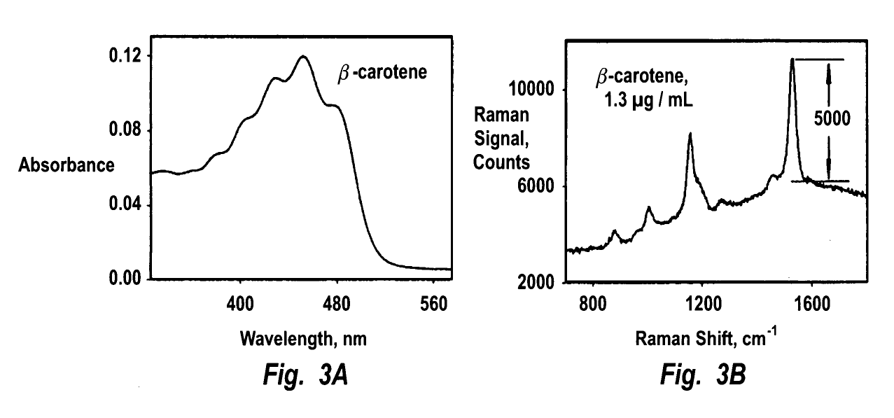

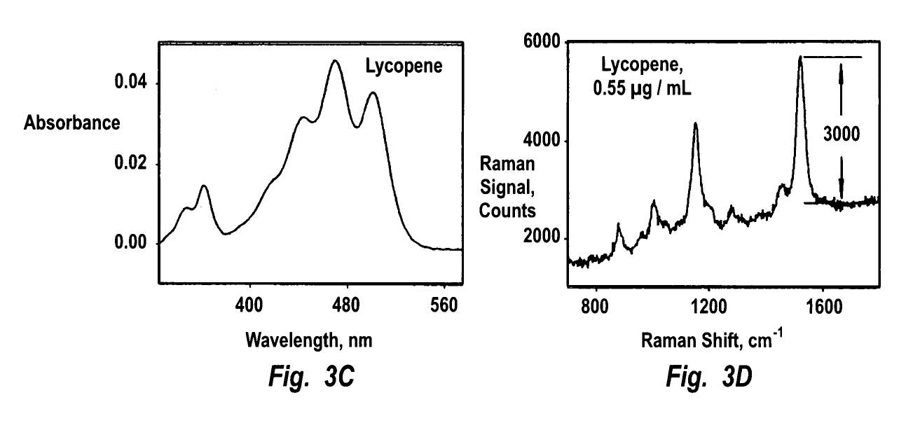

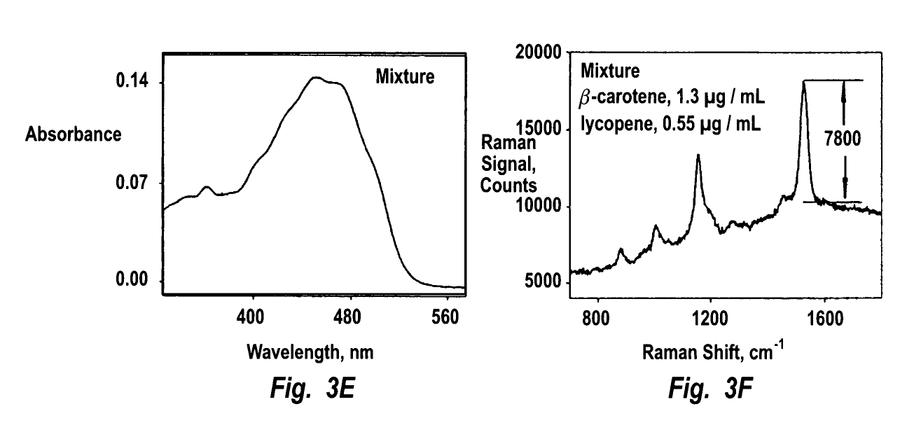

In complex biological tissues several carotenoid species are usually present. For quantification of the composite RRS response it is therefore important to account for individual RRS responses of the excited species. Since the RRS response follows in general the absorption line shape, the individual RRS depends on the extent of the overlap of the excitation laser with the absorption. In the case of equal Raman scattering cross sections, realized when exciting all carotenoids at their respective absorption maxima, the RRS response should add. To verify this assumption, RRS spectra were measured for solutions of kBcarotene, lycopene, and a mixture of both, with 488 nm excitation. The results are shown in the graphs of FIGS. 3A-3F for the solutions, with carotenoid concentrations being higher than typical physiological concentrations encountered in human tissue. It is seen that the RRS response for the carotenoid mixture is roughly equal to the sum of the responses for the individual concentrations. The results demonstrate the capability of resonance Raman spectroscopy to detect a composite response of several carotenoids if excited at the proper spectral wavelength within their absorption bands.

Detection of Macular Pigments

It has been hypothesized that the macular carotenoid pigments, lutein and zeaxanthin, might play a role in the treatment and prevention of age-related macular degeneration (AMD). In the U.S., this leading cause of blindness affects ˜30% of the elderly over age 70. Supportive epidemiological studies have shown that there is an inverse correlation between high dietary intakes and blood levels of lutein and zeaxanthin and risk of advanced AMD. It has also been demonstrated that macular pigment levels can be altered through dietary manipulation and that carotenoid pigment levels are lower in autopsy eyes from patients with AMD.

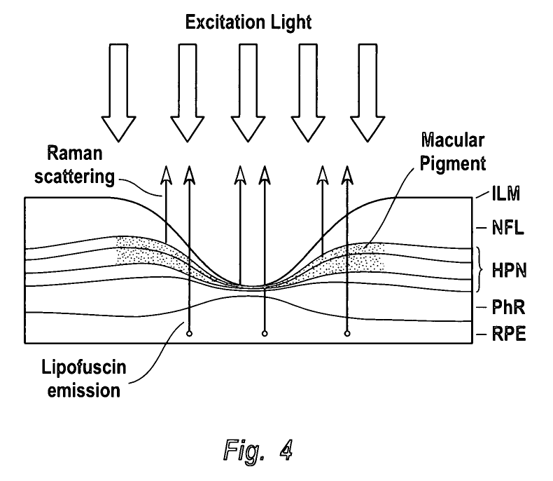

FIG. 4 is a schematic representation of retinal layers that participate in light absorption, transmission and scattering of excitation and emission light. As shown in FIG. 4, the ILM is the inner limiting membrane, the NFL is the nerve fiber layer, the HPN layers are the Henle fiber, plexiform, and nuclear layers, the PhR is the photoreceptor layer, and the RPE is the retinal pigment epithelium. In Raman scattering, the scattering response originates from the MP which is located anteriorly to the photoreceptor layer. The influence of deeper fundus layers such as the RPE is avoided.

Spectroscopic studies of tissue sections of primate maculae (the central 5-6 mm of the retina indicate that there are very high concentrations of carotenoid pigments, shown as shaded area in FIG. 4, in the Henle fiber layer of the fovea and smaller amounts in the inner plexiform layer. The mechanisms by which these macular pigments, derived exclusively from dietary sources such as green leafy vegetables as well as orange and yellow fruits and vegetables, might protect against AMD is still unclear. They are known to be excellent free radical scavenging antioxidants, in a tissue at high risk of oxidative damage due to the high levels of light exposure, and abundant highly unsaturated lipids. In addition, since these molecules absorb in the blue-green spectral range, they act as filters that may attenuate photochemical damage and/or image degradation caused by short-wavelength visible light reaching the retina.

In vivo RRS spectroscopy in the eye takes advantage of several favorable anatomical properties of the tissue structures encountered in the light scattering pathways. First, the major site of macular carotenoid deposition in the Henle fiber layer is on the order of only one hundred microns in thickness. This provides a chromophore distribution very closely resembling an optical thin film having no significant self absorption of the illuminated or scattered light. Second, the ocular media (cornea, lens, vitreous) are generally of sufficient clarity not to attenuate the signal, and they should require appropriate correction factors only in cases of substantial pathology such as visually significant cataracts. Third, since macular carotenoids are situated anteriorly in the optical pathway through the retina (see FIG. 4), the illiuminating light and the backscattered light never encounter any highly absorptive pigments such as photoreceptor (PhR) rhodopsin and retinal pigment epithelium (RPE) melanin, while the light unabsorbed by the macular carotenoids and the forward and side-scattered light will be efficiently absorbed by these pigments.

Autofluorescence Spectroscopy

In contrast, emission of lipofuscin used in autofluorescence-based measurements of MP has to traverse the photoreceptor (PhR) layer (see FIG. 4). In autofluorescence spectroscopy, light emission of deeper fundus layers such as lipofuscin emission from the RPE, can be stimulated on purpose to generate an intrinsic “light source” for single-path absorption measurements of anteriorly located MP layers.

In one method of the invention, autofluorescence (AF) spectroscopy is utilized for MP measurements. As discussed above, in AF spectroscopy, the emission of lipoftiscin is excited at two wavelengths: one wavelength that overlaps both the MP and lipofuscin absorption and another, longer wavelength, that lies outside the MP absorption range but that still excites the lipoftiscin emission. The MP absorption is then derived from the logarithms of the lipofuscin intensity distributions measured in the macula and peripheral retina under both excitations, according to equation (1).

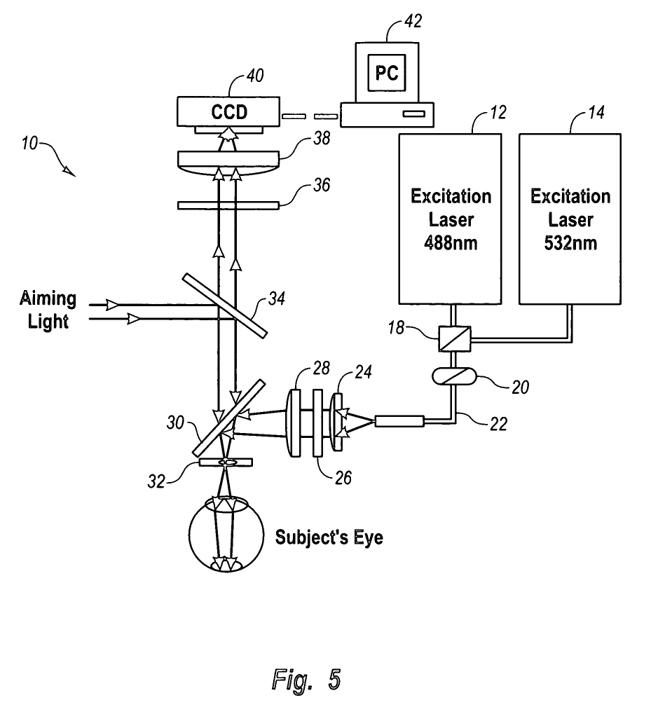

FIG. 5 is a schematic depiction of an apparatus 10 that can be employed for measuring macular pigments using autofluorescence spectroscopy. The apparatus 10 includes a first coherent light source 12, and an optional second coherent light source 14, such as a 488 nm argon laser and an optional 532 nm solid state laser. Alternatively, light sources 12 and 14 may comprise other devices for generating nearly monochromatic light. The light sources 12 and 14 are in optical communication with a light beam delivery means, which can include various optical components in a delivery system for directing laser light to the macular tissue to be measured and directing the emitted light away from the tissue. As shown in FIG. 5, the optical components of the delivery system can include an optical beam combining cube 18, a mechanical shutter/ switch 20, an optical fiber 22, a collimating lens 24, a laser light filter 26, a focusing lens 28, a dichroic beam splitter 30, an aperture 32, a dichroic beam splitter 34, a long-wavelength pass filter 36, and a lens 38.

The light beam delivery system is in optical communication with a detection means such as a light detection system 40, which is capable of measuring the intensity of the scattered light as a function of frequency in the frequency range of interest. The light detection system 40 may comprise, but is not limited to, devices such as a CCD (charge coupled device) camera or detector array, an intensified CCD detector array, a photomultiplier apparatus, photodiodes, or the like.

The detected light is converted by light detection system 40 into a signal which is sent to a quantifying means such as a personal computer 42 or the like. The signal is then analyzed and visually displayed on the monitor of computer 42. It should be understood that the light detection system 40 may also convert the light signal into other digital or numerical formats, if desired. The resultant signal intensities may be calibrated by comparison with chemically measured carotenoid levels from other experiments. The computer 42 preferably has data acquisition software installed that is capable of spectral manipulations.

During operation of apparatus 10, laser excitation light from either light source 12 or 14 is routed via optical beam combining cube 18, mechanical shutter 20, optical fiber 22, dichroic beam splitter 30, and aperture 32, to the retina of the eye to be measured. The lenses 24 and 28 image the output face of the optical fiber delivering the laser excitation light onto the retina of the eye to be measured. The notch filter 26 transmits only the laser excitation light. The lipofuscin emission from the retina of the measured eye is transmitted by dichroic beam splitters 30 and 34, and is detected by light detection system 40 such as a CCD camera, after traversing pass filter 36 and lens 38. A red aiming light, serving as a fixation target during the measurement, is projected onto the retina of the eye via dichroic beam splitter 34. The pass filter 36 transmits only the long-wavelength emission of lipofuscin (e.g., at wavelengths larger than about 715 nm). The light detection system 40 then converts the signal into a form suitable for visual display such as on a computer monitor or the like. For example, digital MP images of a subject are indirectly recorded by detecting the lipofuscin fluorescence of the retinal pigment epithelium in its long-wavelength emission range upon sequential excitation with 488 nm and 532 nm light, and the spatial extent of MP and its topographic concentration distribution is obtained by digital image processing according to equation (1).

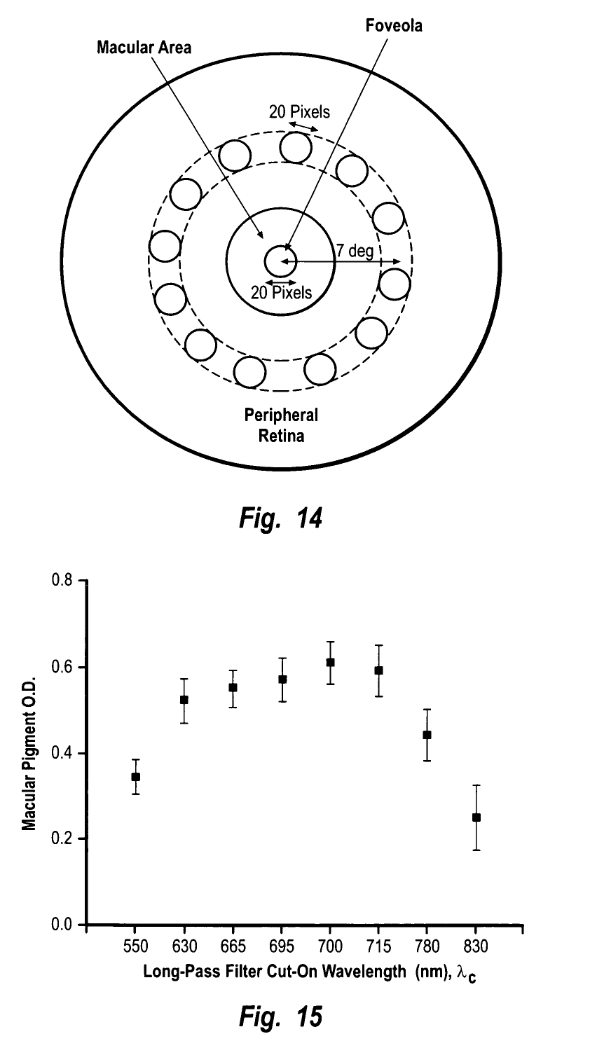

The calculation of the central MP optical density from two measured lipofuscin pixel intensity maps, obtained for 488 and 532 nm excitation, is illustrated in FIG. 14. The MP optical intensity in the center of the macula is determined from these images by calculating the intensities obtained in the various indicated pixel areas (discs with diameter of 20 pixels, chosen in peripheral retina locations and in the center of the macula). In particular, in a first step, for each excitation source, lipofuscin intensities are calculated in the peripheral retina (7 degrees eccentricity) by integrating the pixel intensities inside each of twelve disks located on a circle surrounding the center of the macula (foveola). Each pixel has a width and height of about 20 micrometers. The radius of the circle is 7 degrees, and the diameter of each disk equals 20 pixel widths (about 400 micrometers). The intensities of the twelve disks are then averaged, and a result is obtained for an average lipofuscin intensity in the peripheral retina for 532 nm excitation, Iₐᵥₑ (peri, 532 nm) and an average lipoftiscin intensity for 488 nm excitation, Iₐᵥₑ (peri, 488 nm). In a second step, integrated intensities are calculated for each excitation wavelength for a pixel disk (diameter of 20 pixels) which is centered on the foveola, giving I (fovea, 488 nm) and I (fovea, 532 nm), respectively. The optical density of the MP in the center of the macula is then determined by calculating the expression:
log[I(fovea, 532 nm)/I(fovea, 488 nm)]−log[Iₐᵥₑ(peri, 532 nm)/I(peri, 488 nm)].
Similarly, MP optical densities can be calculated for other regions of the retina by moving the 20 pixel diameter “probe” disk off the center. For example, it is possible, to calculate MP densities along meridional directions, generating line plots of MP versus radial distance from the center of the macula.

Use of the autofluorescence concept to indirectly determine macula pigment must be carried out carefully since this method is not as molecule-specific as Raman spectroscopy. It is assumed in the autofluorescence method that the emission intensity contrast obtained between peripheral retina and central macula is solely due to absorption from MP. However, if any other absorber besides MP exists that contributes to an additional attenuation in the center of the macula, or if there exists any compound contributing fluorescence signals in the macular area, for example, the intensity contrast would be distorted and the contrast would no longer be solely due to MP absorption.

To check for this possibility, the autofluorescence-based MP concentration for a volunteer subject was measured in a series of experiments using long wavelength pass filters with progressively longer cut-on wavelength, i.e., blocking out progressively larger short-wavelength ranges of the lipofuscin fluorescence range. Different MP optical densities are obtained depending on how large of a spectral range of the lipofuscin emission is used in the image registration. If the short or long-wavelength range of the spectrally broad lipofuscin emission band is included in the calculation of the MP densities, lower values for MP optical densities are obtained as compared to the central regions.

If there are no interfering signals to the image contrast, identical MP optical densities for all filters are expected. However, the measurements, shown in FIG. 15, reveal that this is not the case. In the visible wavelength range, up to a filter cut-on wavelength of 630 nm, the MP concentration derived from the image contrast between center and periphery is significantly smaller than that obtained when using at filter cut-on wavelengths above ˜650 nm. This could be caused by a fluorescence signal originating from a compound existing in the path of the excitation light, perhaps from the internal lens. Similarly, there is a decrease of the image contrast at filter cut-on wavelengths above ˜720 nm, on the very long-wavelength shoulder of the lipofuscin emission, which again could be caused by a central fluorescence signal or a peripheral absorption. However, in this extreme long-wavelength emission range, the emission level is only about 10% of the peak emission level. As FIG. 15 shows, the inclusion of this emission range does not produce a significant reduction in image contrast when shorter filter cut-on wavelengths, such as ˜650 nm, e.g., are used that permit the transmission of lipofuscin emission closer to its spectral peak. In conclusion, these results show that in order to obtain maximum intensity contrast between peripheral retina and the center of the macula (leading to maximum MP optical density), the emission wavelength range needs to be limited to the spectral range above about 630 nm where nearly constant MP optical densities are obtained.

Autofluorescence/Raman Spectroscopy

In another method of the invention, both AF spectroscopy and resonance Raman spectroscopy are used to identify and quantify the presence of carotenoids and similar substances in MP. In this combined technique, the AF spectroscopy is used in a similar manner as described above. In using resonance Raman spectroscopy, laser light is directed onto the eye tissue and the scattered light is then spectrally filtered and detected. The scattered light comprises both Rayleigh and Raman scattered light. The Rayleigh light is light which is elastically scattered, which means it is scattered at the same wavelength as the incident laser light. Most of the scattered light is scattered elastically. A small remainder of the light is scattered in an inelastic fashion, and is therefore of different frequencies than the incident laser light. This inelastically scattered light forms the Raman signal. The frequency difference between the laser light and the Raman scattered light, known as the Raman shift, is measured as a difference in wave numbers (or difference in frequencies or wavelengths). The magnitude of the Raman shifts is an indication of the type of chemical present, and the intensities of the Raman signal peaks correspond directly to the chemical concentration.

One of the reasons why Raman spectroscopy is so useful is that specific wave number shifts correspond to certain modes of vibrational or rotational eigenstates associated with specific chemical structures, and hence provide a “fingerprint” of these chemical structures. The Raman shift is independent of the wavelength of incident light used, and hence, in theory, any strong and fairly monochromatic light source can be used in this technique.

The technique of resonance Raman spectroscopy used in the present invention aids in overcoming the difficulties associated with measuring the inherently weak Raman signal. In resonance Raman spectroscopy, a laser source of wavelength near the absorption peaks corresponding to electronic transitions of the molecules of interest is utilized. By making the incident light close to resonant with the electronic absorption frequencies of the molecules of interest, the Raman signal is substantially enhanced, which provides the advantage of being able to use lower incident laser power (which in turn minimizes tissue damage) and also results in less stringent requirements for the sensitivity of the detection equipment.

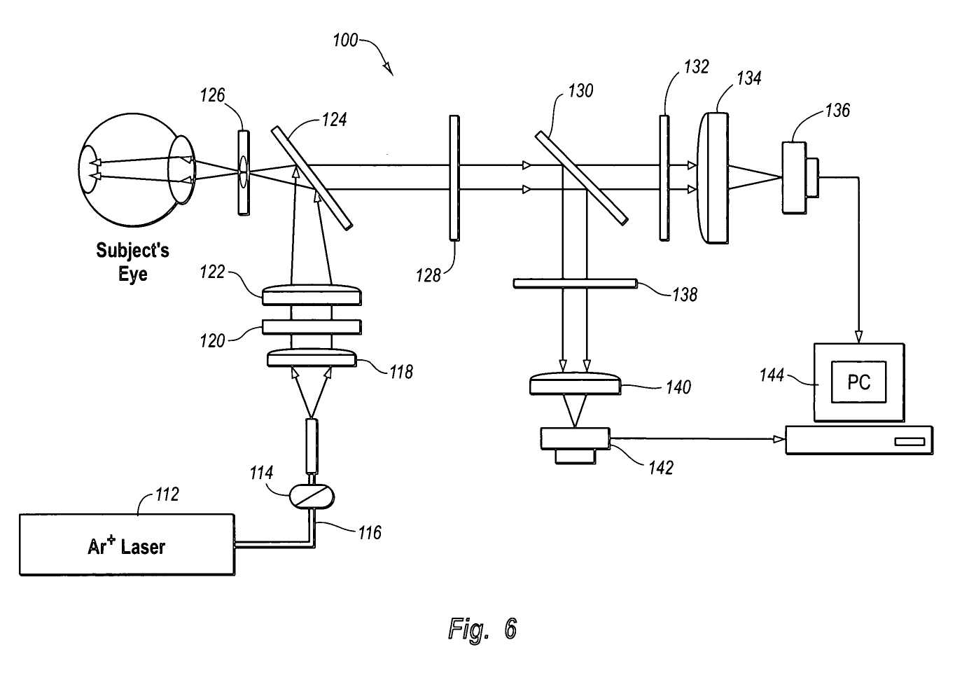

FIG. 6 is a schematic depiction of one embodiment of an apparatus 100 that can be employed for simultaneous Raman and autofluorescence-based imaging of macular pigments. The apparatus 100 includes a light source 112, such as an argon laser. The light source 112 can be configured to generate laser light in a wavelength range from about 450 nm to about 550 nm. Optionally, a second light source can be employed, such as shown for the apparatus of FIG. 5, which provides light at a different wavelength than light source 112 in order to provide more precision if desired.

The light source 112 is in optical communication with a light beam delivery means, which can include various optical components in a delivery system for directing laser light to the macular tissue to be measured and directing the emitted light away from the tissue. As shown in FIG. 6, the optical components of the delivery system can include a mechanical shutter 114, an optical fiber 116, a collimating lens 118, a laser line filter 120, an imaging lens 122, a beam splitter 124 such as a dichroic beam splitter, and an aperture 126. A holographic notch filter 128 is disposed between beam splitter 124 and a beam splitter 130. The beam splitter 130 is placed in the detection path and provides for imaging the MP concentration of a subject's eye with two optical detection channels. A first channel includes a long wavelength pass filter (LWPF) 132 and a focusing lens 134 in optical communication with a first optical detector 136, such as a CCD camera or other optical device, which images the MP via autofluorescence-based detection principles. A second channel includes a transmission Raman filter 138 and a focusing lens 140 in optical communication with a second optical detector 142, such as a CCD camera, which images the MP via Raman spectroscopy. The detected light is converted by the optical detectors into signals that can be analyzed and visually displayed on a monitor of a computer 144.

During operation of apparatus 100, laser excitation light from light source 112 is routed via the delivery system to the retina of the eye to be measured. The lenses 118 and 122 image the output face of the optical fiber delivering the laser excitation light onto the retina of the eye to be measured. The laser line filter 120 transmits only the laser excitation light. The lipofuscin emission from the retina of the measured eye is transmitted by dichroic beam splitters 124 and 130 to the first optical detection channel, and is detected by optical detector 136, after traversing long wavelength pass filter 132 and lens 134. The pass filter 132 transmits only the long-wavelength emission of lipofliscin. The optical detector 136 then converts this signal into a form suitable for imaging on a visual display such as on a computer monitor. The backscattered light containing the Raman signal from the retina of the measured eye is reflected by dichroic beam splitter 130 to the second optical detection channel, and is detected by optical detector 142 after traversing transmission filter 138 and lens 140. The optical detector 142 measures the light intensity at the frequency of the carotenoid Raman peaks of interest, and then converts the Raman signal into a form suitable for imaging on a visual display. The resultant lipofuscin emission and Raman signals are analyzed by computer 144.

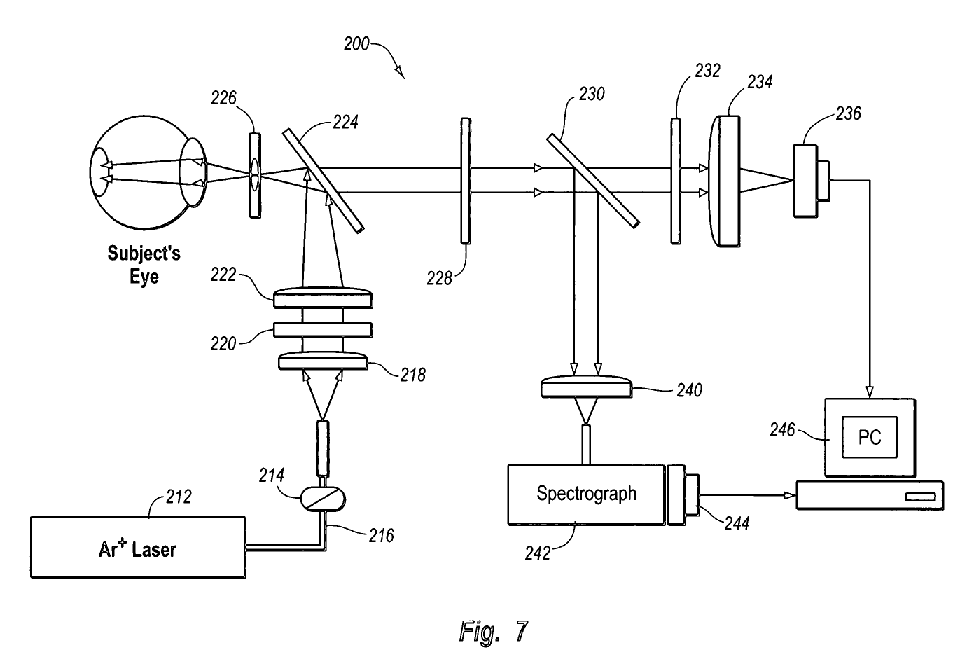

FIG. 7 is a schematic depiction of another embodiment of an apparatus 200 that can be employed for simultaneous Raman and autofluorescence-based detection of macular pigments. The apparatus 200 includes many of the same features as apparatus 100 shown in FIG. 6, including a light source 212, a mechanical shutter 214, an optical fiber 216, a collimating lens 218, a laser line filter 220, an imaging lens 222, a beam splitter 224, and an aperture 226. A holographic notch filter 228 is disposed between beam splitter 224 and a beam splitter 230. The beam splitter 230 is placed in the detection path and provides for imaging the MP concentration of a subject's eye with two optical detection channels. A first channel, which has the same configuration as used in apparatus 100, includes a long wavelength pass filter 232 and a focusing lens 234 in optical communication with an optical detector 236, which images the MP via autofluorescence-based detection principles. A second channel includes a focusing lens 240 in optical communication with a spectrograph device 242 that is operatively connected to an optical detector 244 such as a CCD device. The second optical channel in this embodiment is used for non-imaging, integral Raman detection.

The spectrograph device 242 and optical detector 244 can be selected from commercial spectrometer systems such as a medium-resolution grating spectrometer employing rapid detection with a cooled charge-coupled silicon detector array. For example, a monochromator can be used which employs a dispersion grating with 1200 lines/mm, and a liquid nitrogen cooled silicon CCD detector array with a 25 μm pixel width. Another suitable spectrometer is a holographic imaging spectrometer, which is interfaced with a CCD camera and employs a volume holographic transmission grating.

During operation of apparatus 200, laser excitation light from light source 112 is routed via mechanical shutter 214, optical fiber 216, beam splitter 224, and aperture 226, to the retina of the eye to be measured. The lenses 218 and 222 image the output face of the optical fiber delivering the laser excitation light onto the retina of the eye to be measured. The laser line filter 220 transmits only the laser excitation light. The lipofliscin emission from the retina of the measured eye is transmitted by beam splitter 230 to the first optical detection channel and is detected by optical detector 236, which converts the signal into a form suitable for imaging on a computer monitor. The backscattered light containing the Raman signal from the retina of the measured eye is reflected by beam splitter 230 to the second optical detection channel, and is detected by optical detector 244 after traversing lens 240 and spectrograph device 242. The resultant lipofuscin emission and Raman signals are analyzed by a computer 246.

The following examples are given to illustrate the present invention, and are not intended to limit the scope of the invention.

### EXAMPLE 1

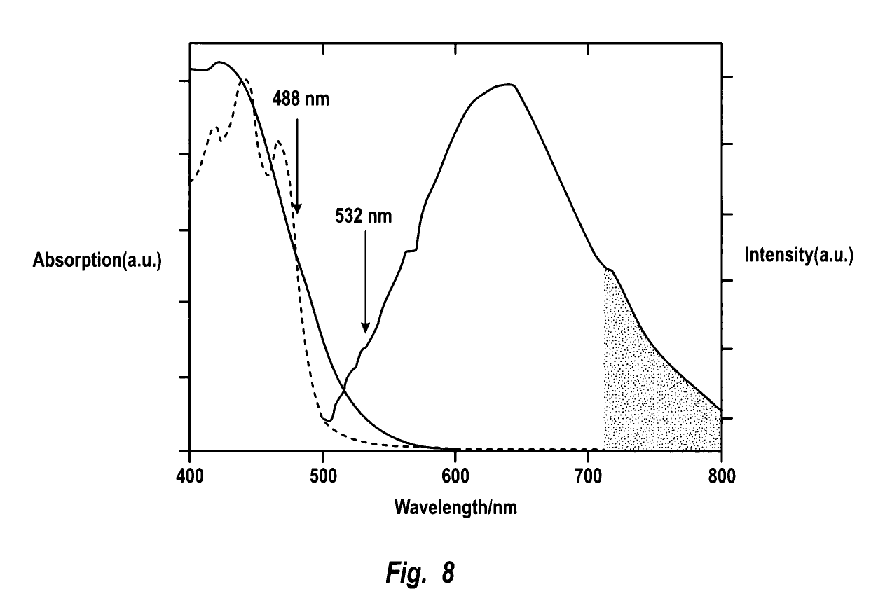

FIG. 8 is a graph of the absorption (solid curve at left) and emission spectra (solid curve at right) of A2E, the main fluorophore of lipofuscin, dissolved in methanol. The absorption peaks in the blue spectral range at about 430 nm, and the emission in the far red spectral range at about 650 nm. The absorption of the macular pigments lutein and zeaxanthin is also indicated, as a dotted line, and shows that it essentially occurs in the same spectral range as that of lipofuscin. Two spectral positions of laser excitation lines, 488 nm and 532 nm, respectively, are shown as arrows. The 488 nm line is seen to overlap both the lipofuscin and the MP absorption on the long wavelength shoulder. The 532 nm line is outside the spectral absorption range of MP but overlaps that of lipofliscin. The vertical line at 715 nm indicates the wavelength where a long-wavelength pass filter, used in the measurement of lipofuscin emission, has reached transparency, limiting the detection of the lipofuscin emission intentionally only to wavelengths beyond ˜700 nm (shown as gray shaded area).

### EXAMPLE 2

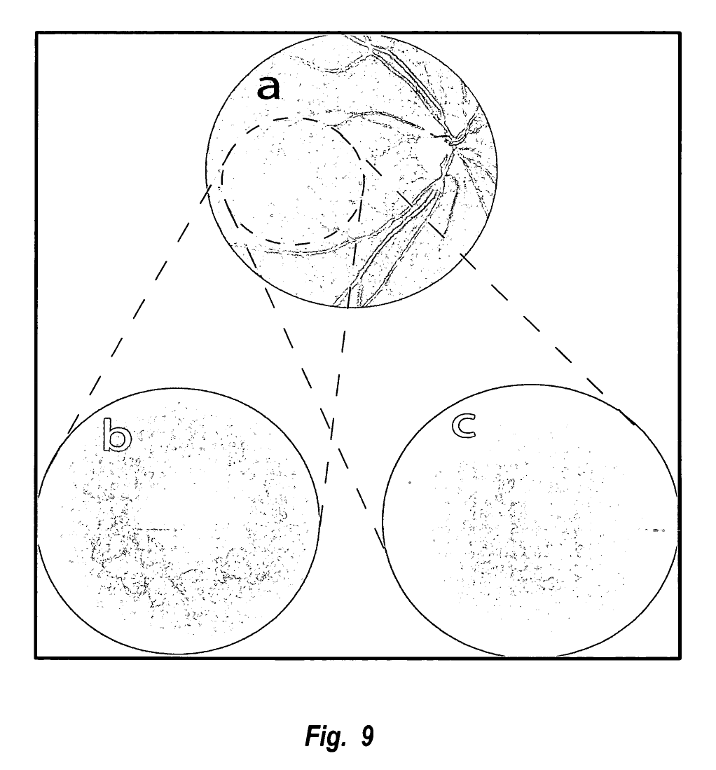

FIG. 9 includes photomicrographs of the retina of a human volunteer subject, showing image a obtained by measuring the reflection of white light (standard fundus image), image b which is a lipofuscin fluorescence digital fundus image obtained under 488 nm excitation, and image c which is the lipofuscin fluorescence digital fundus image obtained under 532 nm excitation. Images b and c were obtained by detecting lipofuscin fluorescence in its long-wavelength emission range (λ>700 nm). The field of view for image a is larger than for images b and c in order to illustrate the relative location of the macular region (gray shaded area on left side of image a) with respect to the optic nerve disk (bright white spot on right side of image a). Images b and c are centered on the macular region and are recorded, respectively, with 488 nm light that is absorbed by both lipofuscin and macular pigments, and with 532 nm light that falls outside the absorption range of macular pigments, and therefore only weakly excites the lipofuscin emission. A digital subtraction image due only to the MP absorption can be obtained by subtracting image c from image b. For example, the spatial extent of MP and its topographic concentration distribution can be obtained by digitally subtracting image c, serving as a reference pixel intensity map, from image b, which has pixel areas with reduced intensities due to absorption of the lipofuscin emission by MP (central shaded area).

### EXAMPLE 3

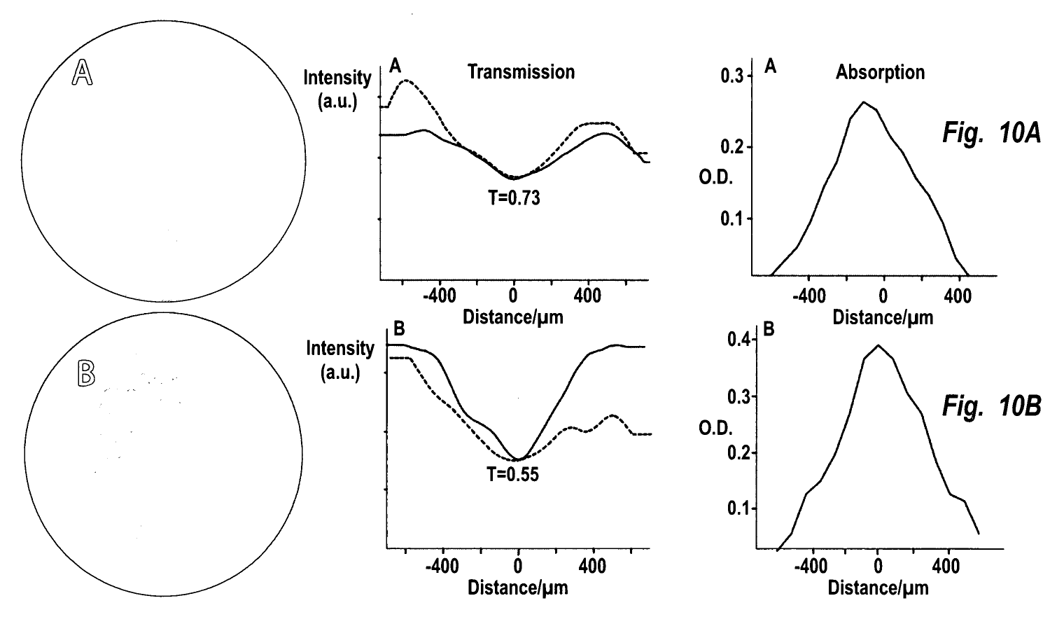

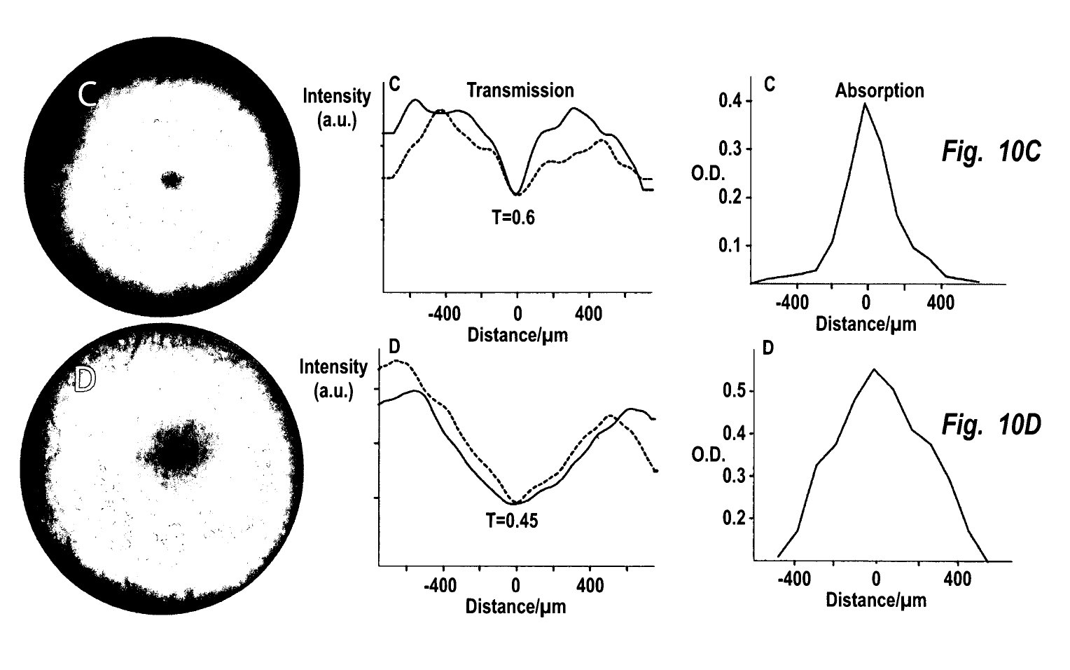

FIG. 10 includes photomicrographs of the retina of four human volunteer subjects (A-D), showing digital subtraction images of gray-scale coded spatial MP distributions integrated over the macular region obtained from the subjects A-D. Corresponding line plots of transmissions (intensity, a.u. vs. distance/tm) and line plots of absorptions (optical density, O.D. vs. distance/pm) derived from the subtraction images by evaluating the corresponding pixel intensities along horizontal meridional horizontal lines of the images are also presented in FIG. 10. As shown in FIG. 10, the spatial width, symmetries, and concentrations of MP vary significantly in subjects C and D. For example, subjects C and D had large differences of MP regarding spatial extent (small in C and large in D).

### EXAMPLE 4

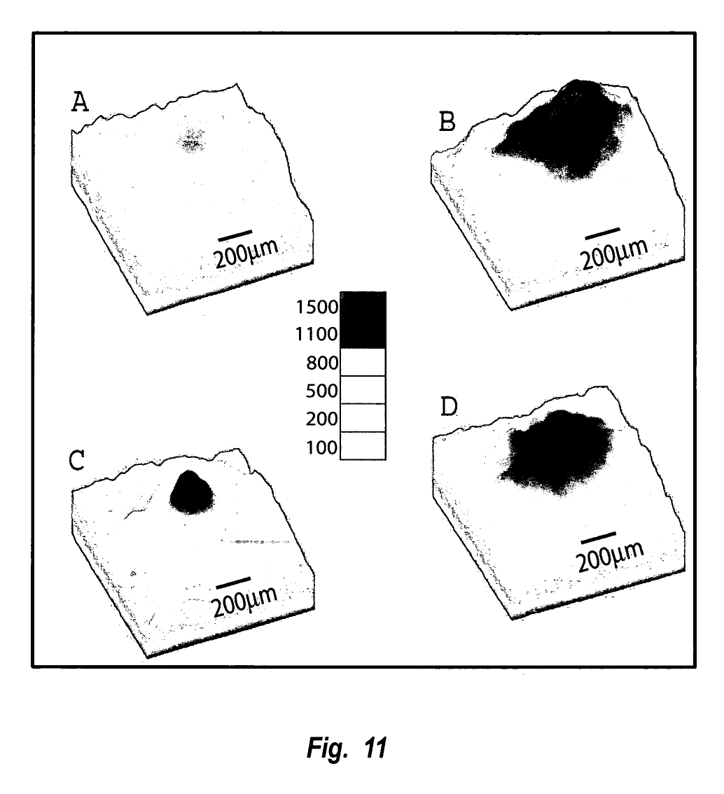

FIG. 11 displays pseudocolor topographical maps showing MP distributions in four volunteer subjects A-D. The MP concentrations vary according to the pseudocolor bar code shown in FIG. 11. As depicted in FIG. 1 1, large spatial and concentration variation of pigments were present between subjects A-D.

### EXAMPLE 5

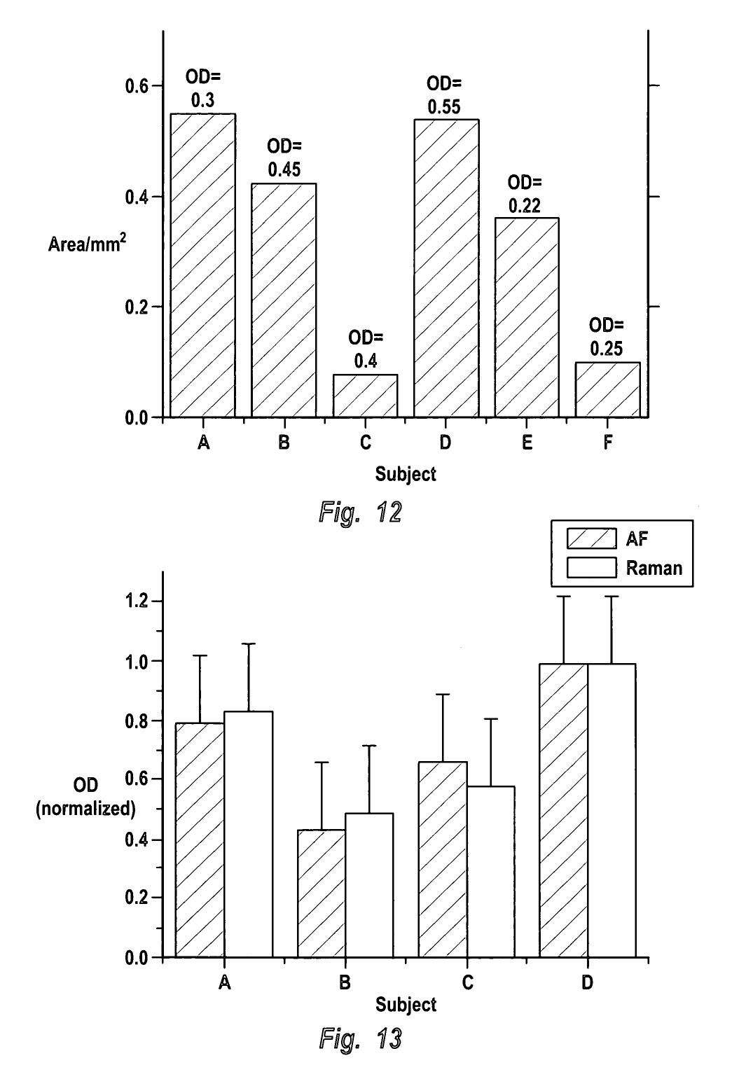

FIG. 12 is a bar graph of MP concentrations for six individuals (subjects A-F), showing the total pigment concentration of each individual, obtained by integrating each individual's distribution over its area. Total concentrations obtained in this way can be compared with concentration values measured by integral Raman detection.

### EXAMPLE 6

FIG. 13 is a bar graph showing a comparison of MP intensities, measured for four subjects A-D by autofluorescence and resonance Raman detection techniques. The bars for the Raman responses (open bars) and autofluorescence responses (hatched bars) are integrated over the macular region. The bar heights obtained for each individual with either technique are very similar, indicating that both techniques appear to quantitate the macular pigment concentrations in different individuals in a consistent fashion.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for measuring macular pigments, comprising:
providing a first light source and an second light source that emit different wavelengths of light;
directing light from the first light source onto macular tissue of an eye for which macular pigment levels are to be measured, the light from the first light source having an intensity that does not substantially alter macular pigment levels in the macular tissue;
directing light from the second light source onto macular tissue of the eye, the light from the second light source having an intensity that does not substantially alter macular pigment levels in the macular tissue;
collecting light emitted from the macular tissue, the collected light comprising lipoftiscin emission from the macular tissue at two wavelengths, including a first excitation wavelength that overlaps both the macular pigment V and lipofuscin absorption range, and a second excitation wavelength that is longer than the first excitation wavelength and lies outside the macular pigment absorption range but still excites lipofuscin emission;
quantifying the lipoftiscin emission intensities obtained with the first and second excitation wavelengths; and
determining the macular pigment levels in the macular tissue from the differing lipofuscin emission intensities in the macula and peripheral retina.

2. The method of claim 1, wherein the first light source generates coherent light at a wavelength of about 488 nm.

3. The method of claim 1, wherein the second light source generates coherent light at a wavelength of about 532 nm.

4. The method of claim 1, wherein the first and second excitation wavelengths of the lipofuscin emission are from fluorescence of the retinal pigment epithelium of the eye upon sequential excitation with the light from the first and second light sources.

5. The method of claim 4, wherein the fluorescence of the retinal pigment epithelium is used to produce digital macular pigment images of the macular tissue.

6. The method of claim 5, further comprising obtaining spatial extent and topographic concentration distribution of the macular pigments by digital image subtraction.

7. The method of claim 1, wherein the macular tissue resides in a live subject.

8. An apparatus for measuring macular pigments, comprising:
a first light source that generates light at a first wavelength;
an optional second light source that generates light at a second wavelength that is different from the first wavelength;
delivery means for directing light sequentially from the first and second light sources onto macular tissue of an eye for which macular pigment levels are to be measured;
detection means for collecting light emitted from the macular tissue, the collected light comprising lipofuscin emission from the macular tissue at two excitation wavelengths; and
quantifying means for determining intensities of the lipofuscin emission at the excitation wavelengths, and determining the macular pigment levels in the macular tissue from the differing lipofuscin emission intensities in the macula and peripheral retina.

9. The apparatus of claim 8, wherein the first light source generates coherent light at a wavelength of about 488 nm.

10. The apparatus of claim 8, wherein the second light source generates coherent light at a wavelength of about 532 nm.

11. The apparatus of claim 8, wherein the delivery means comprises a series of optical components configured to direct light into and away from the macular tissue of the eye.

12. The apparatus of claim 8, wherein the detection means comprises a device selected from the group consisting of a CCD camera, a CCD detector array, an intensified CCD detector array, a photomultiplier apparatus, and photodiodes.

13. The apparatus of claim 8, wherein the quantifying means comprises a personal computer.

14. A method for measuring macular pigments, comprising:
providing at least one light source that generates light at a wavelength that produces an autofluorescence lipofuscin emission and a Raman response with a wavelength shift for carotenoids to be detected;
directing light from the light source onto macular tissue of an eye for which macular pigment levels are to be measured, the light from the light source having an intensity that does not substantially alter macular pigment levels in the macular tissue;
collecting light emitted from the macular tissue in a first optical channel, the collected light in the first optical channel comprising lipofuscin emission from the macular tissue at two wavelengths, including a first excitation wavelength that overlaps both the macular pigment and lipofuscin absorption range, and a second excitation wavelength that is longer than the first excitation wavelength and lies outside the macular pigment absorption range but still excites lipofuscin emission;
quantifying the lipofuscin emission intensities at the first and second excitation wavelengths;
determining the macular pigment levels in the macular tissue from the differing lipofuscin emission intensities in the macula and peripheral retina;
collecting light scattered from the macular tissue in a second optical channel, the scattered light in the second optical channel including elastically and inelastically scattered light, the inelastically scattered light producing a Raman signal corresponding to carotenoids in the tissue;
filtering out the elastically scattered light; and
quantifying the intensity of the Raman signal.

15. The method of claim 14, wherein the light source generates laser light in a wavelength that overlaps absorption bands of the carotenoids to be detected.

16. The method of claim 14, wherein the light source generates laser light in a wavelength range from about 450 nm to about 550 nm.

17. The method of claim 14, wherein the light source generates laser light at a wavelength of about 488 nm.

18. The method of claim 14, further comprising a second light source that generates laser light at a wavelength of about 532 nm.

19. The method of claim 14, wherein the first and second wavelengths of the lipofuscin emission are from fluorescence of the retinal pigment epithelium of the eye.

20. The method of claim 19, wherein the fluorescence of the retinal pigment epithelium is used to produce digital macular pigment images of the macular tissue.

21. The method of claim 20, further comprising obtaining spatial extent and topographic concentration distribution of the macular pigments by digital image subtraction.

22. The method of claim 14, wherein the scattered light is measured at frequencies characteristic of macular carotenoids.

23. The method of claim 14, wherein the Raman signal is quantified via signal intensity calibrated with actual carotenoid levels.

24. The method of claim 14, wherein the macular tissue resides in a live subject.

25. An apparatus for measuring macular pigments, comprising:
at least one light source that generates light at a wavelength that produces an autofluorescence lipofuscin emission, and a Raman response with a wavelength shift for carotenoids to be detected;
a first optical channel;
a second optical channel;
delivery means for directing light from the autofluorescence lipofuscin emission to the first optical channel, and directing scattered light containing a Raman signal to the second optical channel;
a first optical detector for collecting light from the first optical channel;
a second optical detector for collecting light from the second optical channel; and
quantifying means for determining intensities of the lipofuscin emission from the first optical channel, and determining Raman signal intensity of the scattered light from the second optical channel.

26. The apparatus of claim 25, wherein the light source generates laser light in a wavelength that overlaps absorption bands of the carotenoids to be detected.

27. The apparatus of claim 25, wherein the light source generates laser light in a wavelength range from about 450 nm to about 550 nm.

28. The apparatus of claim 25, wherein the light source generates laser light at a wavelength of about 488 nm.

29. The apparatus of claim 25, further comprising a second light source that generates laser light at a wavelength of about 532 nm.

30. The apparatus of claim 25, wherein the delivery means comprises a series of optical components configured to direct light into and away from macular tissue of an eye.

31. The apparatus of claim 25, wherein the first and second optical detectors are selected from the group consisting of a CCD camera, a CCD detector array, an intensified CCD detector array, a photomultiplier apparatus, and photodiodes.

32. The apparatus of claim 25, wherein the second optical channel is in optical communication with a spectrographic device that is operatively connected to the second optical detector.

33. The apparatus of claim 32, wherein the second optical channel is configured for non-imaging, integral Raman detection.

34. The apparatus of claim 25, wherein the quantifying means comprises a personal computer.
